(19) United States
(12) Patent Application Publication
(10) Pub. No.: US 2024/0265542 A1
(43) Pub. Date: Aug. 8, 2024

(54) THREE-DIMENSIONAL ARTIFICIAL INTELLIGENCE-AIDED CLASSIFICATION SYSTEM FOR GLAUCOMATOUS OPTIC NEUROPATHY AND MYOPIC OPTIC DISC MORPHOLOGY FROM OPTICAL COHERENCE TOMOGRAPHY SCANS

(71) Applicant: The Chinese University of Hong Kong, Hong Kong (CN)

(72) Inventors: Carol Yim Lui CHEUNG, Hong Kong (CN); Clement Chee Yung THAM, Hong Kong (CN); Anran RAN, Hong Kong (CN); Pheng Ann HENG, Hong Kong (CN); Xi WANG, Hong Kong (CN)

(21) Appl. No.: 18/163,359

(22) Filed: Feb. 2, 2023

(51) Int. Cl. G06T 7/00; A61B 3/00; A61B 3/10; A61B 3/12; G06V 10/764; G16H 30/40; G16H 50/20

(52) U.S. Cl. CPC G06T 7/0014; A61B 3/0025; A61B 3/102; A61B 3/12; G06V 10/764; G16H 30/40; G16H 50/20; G06T 2200/04; G06T 2200/24; G06T 2207/10101; G06T 2207/20081; G06T 2207/30041; G06T 2207/30168; G06V 2201/03

(57) ABSTRACT

The subject invention pertains to an artificial intelligence-aided classification system for glaucomatous optic neuropathy (GON) and myopic optic disc morphology (myopic features, MF) from three-dimensional (3D) optical coherence tomography (OCT) scans, which includes a deep-learning (DL) based “pre-diagnosis model” for image quality control and a multi-task DL-based classification and visualization model for GON and MF detection, including heatmaps for visualizing the identified features. The invention provides an Al-platform with the integration of developed 3D DL algorithms, an information management system, connecting to a commercially available OCT device. This Al-platform includes a user interface for real-time OCT image extraction, input data configuration, image uploading, images analysis via a graphics processing unit (GPU) server, and Al reports generation. The platform provides outputs including image quality, GON classification, MF classification, AI scores, and referral suggestion.

### BACKGROUND OF THE INVENTION

Glaucoma is the leading cause of visual morbidity and blindness worldwide, and it is projected to affect 111.8 million people by 2040. Visual loss from glaucoma is currently irreversible even with treatments, and early to moderate glaucoma is largely asymptomatic, because the visual loss usually starts from the periphery and slowly progresses towards the center. Prompt and accurate detection of glaucoma is extremely important in inhibiting and reducing irreversible visual impairment and blindness. Optical coherence tomography (OCT), a non-contact and non-invasive imaging technology for cross-sectional and three-dimensional (3D) view of the retina and optic nerve head (ONH), is now commonly used to evaluate the structural changes of glaucoma, i.e., glaucomatous optic neuropathy (GON, also referred to as “glaucoma” herein). OCT is widely used to quantify retinal nerve fiber layer (RNFL), neuro-retinal rim, and other inner retinal layers (e.g., ganglion cell layer, inner plexiform layer). OCT is sensitive and specific for detecting GON, especially when combined with other ophthalmoscopic modalities.

However, poor scan quality due to patients' poor co-operation, operators' skills, or device-dependent factors (e.g., inaccurate optic disc margins delineation) can affect the metrics generated from the OCT. Conventionally, for commercial systems (e.g., the Cirrus High-Definition OCT, Carl Zeiss Meditec, Dublin, CA, USA), signal strength (SS) is the main parameter to include or exclude OCT scans for further quantitative analysis. Image quality is indicated by SS ranging from 0 (worst quality) to 10 (best quality), representing the average of signal intensity of OCT volumetric scans, and scans with SS of 6 or above are often defined as sufficient for the further analysis. However, even with acceptable SS, it is still hard to assess other OCT image quality issues, such as off-centration, out of registration, signal loss, motion artifacts, mirror artifacts, or blurriness of OCT volumetric data. Such image quality assessment indeed requires highly trained operators and interpreters with specialized knowledge in OCT which is a big challenge due to the lack of manpower in clinics. Besides, it is impractical for human assessors to grade every OCT volumetric scan which can be a time-consuming and tedious process, particularly in busy clinics.

In addition, myopic optic disc morphology (also referred to as “myopic features” or, “MF” herein), such as peripapillary atrophy (PPA) and optic disc tilting, also influences GON identification based on RNFL thickness measurement alone, which should be taken into account when interpreting the optic disc and its circumpapillary regions for diagnosis. For example, PPA beta zone correlates with glaucoma, while gamma zone is related to axial globe elongation. A higher degree of vertical optic disc tilting is associated with a more temporally positioned RNFL thickness peak. Eyes with longer axial length are associated with significantly higher percentages of false-positive errors based on an OCT built-in normative database. Hence, evaluating glaucoma structural changes using OCT based on RNFL thickness and built-in normative databases alone may not be reliable. MF can also result in thinning of RNFL thickness (i.e., outside of the normal RNFL range) in eyes without glaucoma structural changes. Other diagraphs and metrics, such as topographical ONH measurements, RNFL thickness map, RNFL deviation map, and circumpapillary RNFL thickness with “double-hump pattern” should also be evaluated to differentiate these two pathologies carefully. For example, in purely myopic eyes, the “double-hump pattern” can be present but with temporal shift due to optic disc tilting. The RNFL thickness map also shows normal thickness except that the angle between superior and inferior RNFL bundles is smaller. While in eyes with glaucoma, RNFL “double-hump pattern” is altered and thinner RNFL thickness appears at specific regions. Thus, interpretation of the results requires experienced glaucoma specialists or highly trained assessors who have good knowledge on both glaucoma and OCT limitations.

### BRIEF SUMMARY OF THE INVENTION

Embodiments of the subject invention provide three-dimensional (3D) artificial intelligence (AI)-aided classification systems and methods for glaucomatous optic neuropathy (GON) and myopic optic disc morphology (MF) from optical coherence tomography (OCT) scans. Embodiments provide a novel method of building an AI platform integrating an information management system, an AI image analysis system, and a user interface. In certain embodiments the image analysis system includes image quality assessment, GON detection, and MF detection. Embodiments provide systems and methods for deep learning (DL), composed of multiple processing layers, that allows computational models to learn representative features with multiple levels of abstraction. Embodiments provide models useful in pattern recognition and image analysis. Embodiments provide a 3D AI-aided automated image analysis for glaucoma and myopia structural changes classification from volumetric OCT scans, which includes a DL-based pre-diagnosis model developed by SE-ResNeXt in 3D version for image quality control (e.g., providing an outcome of “sufficient” or “insufficient”) and a multi-task DL-based classification model developed by ResNet-37 in 3D version for glaucoma and myopia structural changes (e.g., providing outcomes of “Yes GON” or “No GON” and “Yes MF” or “No MF” with AI scores). Embodiments generate heatmaps using class activation map for visualizing the identified features.

Embodiments of the subject invention provide an AI-platform with the integration of developed 3D DL models (e.g., an image quality control model, a multi-task model for glaucoma and myopic features detection), an information management system (e.g., a graphic processing unit server enabling rapid data storage, search, and retrieval), and a commercially available OCT device (e.g., Cirrus HD-OCT which can directly export raw volumetric data and XML file for data extraction and analysis, or other devices now known or later developed). Embodiments of the provided AI-platform include a user interface (front-end and back-end) for real-time OCT image extraction, input data configuration (e.g., subject study ID, exam date, age, gender, imaging protocol), image uploading, images analysis via a graphics processing unit (GPU) server, and AI reports generation. Embodiments provide AI reports with outputs including but not limited to image quality, glaucoma classification, myopic features classification, AI scores, and referral suggestions rapidly (e.g., within 5 minutes for a typical clinical imaging data set.)

### BRIEF DESCRIPTION OF THE DRAWINGS

FIGS. 1A-1B show examples of correctly detected eyes with glaucoma and eyes without glaucoma detected by a 3D DL model according to an embodiment of the subject invention, the corresponding heatmaps generated by Class Activation Map (CAM), and the paired enface fundus images. (1A) Depicts a correctly detected OCT volumetric scan as “yes glaucoma” from the cross-sectional (left side images) and top (top-right images) view as well as the paired enface 2D fundus (bottom-central image.) (1B) Depicts a correctly detected OCT volumetric scan as “no glaucoma” from the cross-sectional (left side images) and top (top-right images) view as well as the corresponding enface 2D fundus (bottom-central image.)

FIGS. 2A-2B show examples of truly detected eyes by a multi-task 3D DL model according to an embodiment of the subject invention. The heatmaps shows in (2A) an eye with myopic features (e.g., peripapillary atrophy) the optic disc area, and the areas with PPA is red-orange colored. In (2B) is shown an eye without myopic features, only the optic disc was red-orange colored.

FIGS. 3A-3B show image quality assessment using a squeeze-and-excitation (SE)-ResNeXt model in 3D version according to an embodiment of the subject invention. (3A) Shows the architecture of the model with SE-ResNeXt building blocks. (3B) Details the architecture of SE-ResNeXt building blocks. SE=squeeze-and-excitation. BN=batch normalization, GAP=global average pooling, Conv=convolutional, Avg=average.

FIG. 4 is a diagram showing the structure of a 3D multi-task deep learning model according to an embodiment of the subject invention.

FIG. 5 is a diagram showing the user interface for image quality assessment, GON and MF detection according to an embodiment of the subject invention.

FIGS. 6A-6B show the workflow and an AI report generated by the intervention (i.e., the 3D AI-aided classification systems for GON and MF from OCT scans, which is an AI platform integrating an information management system, an AI image analysis system, and a user interface) according to an embodiment of the subject invention.

### DETAILED DISCLOSURE OF THE INVENTION

Embodiments of the subject invention provide systems and methods for a novel 3D AI-aided classification system for GON and MF from OCT volumetric scans with additional integrated image quality control. Embodiments exhibit numerous advantages, including but not limited to robust application across disparate datasets and populations, integrated DL techniques, straightforward (e.g., yes/no) outputs facilitate rapid screening, improved performance from 3D volumetric scanning, integrated platform from user interface through AI to output, direct integration to bring AI analysis to commonly available imaging platforms, and further integration with additional deep learning models for the detection of additional disease states.

Embodiments provide a novel method of building an AI platform integrating an information management system, an AI image analysis system, and a user interface. The image analysis system includes image quality assessment, GON detection, and MF detection.

In certain embodiments the development and testing datasets can be collected from multiple eye centers from different countries and regions including different ethnic backgrounds. Embodiments have performed consistently well in all tested datasets. The training-tuning curves also illustrated that the proposed DL model was not overfitted. Thus, embodiments can be applied on other unseen datasets, even among different populations.

Embodiments provide state-of-the-art DL techniques such as irrelevancy reduction and self-attention mechanism for the image quality control task, and multi-task technique for the disease detection task. Irrelevancy reduction omits the parts of irrelevant signals that should not be noticed by the signal receiver, which can improve the AI performance for image quality control. In certain embodiments denoising is provided to reduce the irrelevancies of OCT scans since the noise of OCT scans can impeded the medical analysis either visually or programmatically. For denoising, embodiments provide nonlocal means which can be performed both vertically (along x, z facets) and horizontally (along x, y facets) with different sets of parameters. Vertically, the template window size can be set to 10, whereas the search window size can be set to 5 with a filter strength of 5. Horizontally, the template window size can be set to 5, and search window size can be set to 5 with a filter strength of 5. The self-attention mechanism is provided to help the model recognize the more important areas and extract features automatically in the original OCT volumetric scans. Multi-task learning is provided as a training paradigm to train DL models with data from multiple tasks simultaneously, using shared representations to learn the common features between a collection of related tasks, which provides advantages of integrating information across domains and extracting more general features for different tasks.

Embodiments generate straightforward output of Yes/No GON and Yes/No MF with automated image quality assessment, which can further strengthen OCT as a screening tool in settings without sufficient ophthalmologists experts on site as it provides a clear AI report with outcomes of image quality (“sufficient” or “insufficient”), GON detection (“Yes GON” or “No GON”), MF detection (“Yes MF” or “No MF”) with AI scores, and referral suggestions. End users, such as primary care technicians, optometrists, or family physicians, can interpret the AI report easily.

Embodiments analyze 3D OCT scans and show generally better performance than related art 2D models analyzing cross-sectional 2D B-scans for both GON and MF detection. OCT receives tissue information in depth by measuring the magnitude and echo delay of backscattered light. Cross-sectional images are generated by transversely scanning the incident optical beam and performing axial scans, named B-scan. Volumetric scans can be generated by raster scanning a series of cross-sectional images (i.e., B-scans). For certain type of commercialized OCT device (e.g., Cirrus OCT), there can be 200 or 128 B-scans in each volumetric scan depending on the imaging protocol. Embodiments provide volume-level output instead of B-scan level output which is more straightforward for physicians (e.g., non-ophthalmologists) to interpret the results and requires less manpower or computation power to deal with a required large number of B-scans.

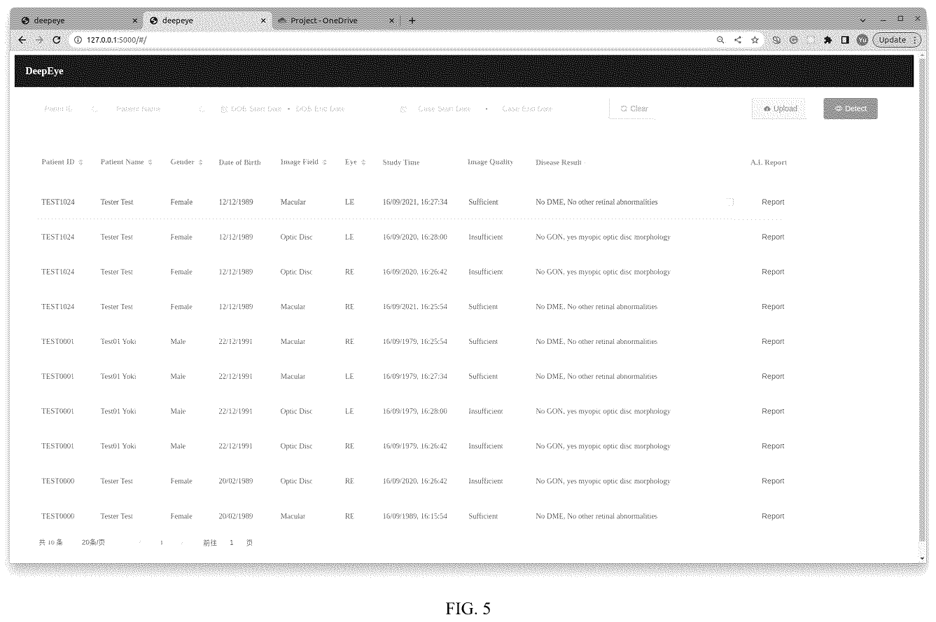

Embodiments of the subject invention provide an AI-platform that includes a user interface (e.g., front-end and back-end) for real-time OCT image extraction, input data configuration (e.g., subject study ID, exam date, age, gender, imaging protocol), image uploading, and images analysis via a graphics processing unit (GPU) server, and AI reports generation. Embodiments provide outputs including image quality, glaucoma classification, and myopic features classification within a few minutes. In certain embodiments the AI-platform is implemented into a commercial OCT device and configured to automatedly detect the exported data for further image analysis and disease detection (FIG. 5).

In certain embodiments the AI-platform integrates additional DL models using OCT macula volumetric scans to detect other diseases such as diabetic macula edema in the future (FIG. 5).

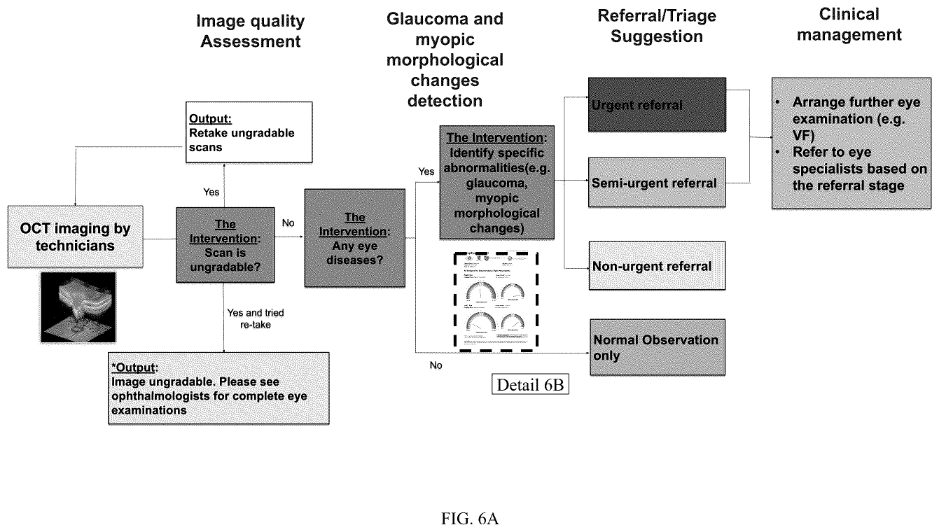

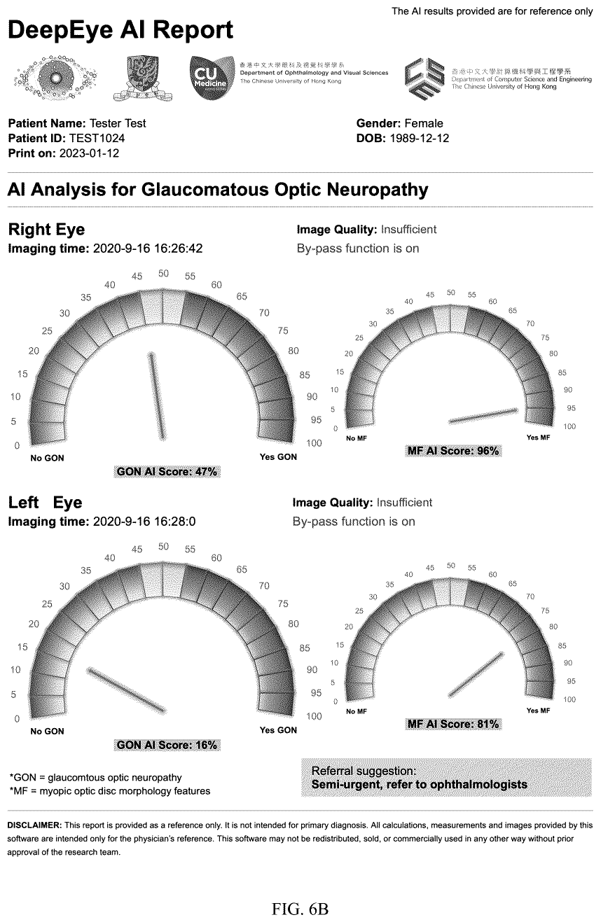

Certain embodiments of the subject invention provide algorithms, programs, systems, or methods for identifying GON and MF by pre-diagnosis image quality control and ensuring the gradeability of OCT scans by providing an immediate onsite assessment of image quality. This can allow retaking of OCT, if necessary, of subjects within the same visit and also reduce the expertise required in collecting OCT images (FIGS. 6A and 6B).

Embodiments of the subject invention provide improved medical care to millions of patients at risk for glaucoma (e.g., elderly patients, 65 years and over) to inhibit or prevent irreversible vision loss, with faster and more reliable screening delivered at a reduced overall cost. These benefits are multiplied when patients are recommended for re-screening every two years.

The inventors have tested the image quality control and glaucoma and myopia structural changes DL modules in retrospective multi-center cohorts. The performance results are shown in Table 1, and Table 2. Embodiments of the subject invention provide an AI-platform to integrate the provided DL modules with an information management system for clinical deployment with an OCT device.

TABLE 1
Performances of the image quality control deep learning
model. (AUROC = the area under the receiver operating
characteristic curve, CI = confidence interval.)

| | | Sensitivity, | Specificity, | Accuracy, |
|---|---|---|---|---|
| | AUROC | % | % | % |
| | (95% CI) | (95% CI) | (95% CI) | (95% CI) |
| Internal | 0.954 | 86.2% | 92.6% | 91.0% |
| validation | (0.938-0.970) | (80.0-92.4) | (86.8-96.9) | (87.3-93.5) |
| External | 0.816 | 69.1% | 81.3% | 78.2% |
| validation 1 | (0.780-0.852) | (58.0-84.0) | (64.0-89.4) | (68.8-82.7) |
| External | 0.857 | 78.3% | 82.8% | 82.6% |
| validation 2 | (0.800-0.914) | (61.7-91.7) | (71.9-94.6) | (74.2-89.9) |

TABLE 2
The discriminative performance of the multi-task 3D deep learning model for
detecting glaucomatous optic neuropathy (GON) and myopic optic disc morphology
(also referred to herein as myopic features, MF) in all datasets. (AUROC =
the area under the receiver operating characteristic curve, CI = confidence
interval, PPV = positive predictive value, NPV = negative predictive value)

| | | | | | |
|---|---|---|---|---|---|
| | Sensitivity | Specificity | Accuracy | PPV | NPV |
| AUROC | % | % | % | % | % |
| (95% CI) | (95% CI) | (95% CI) | (95% CI) | (95% CI) | (95% CI) |

Internal validation

| | | | | | |
|---|---|---|---|---|---|
| GON 0.949 | 88.0 | 91.6 | 89.4 | 92.2 | 87.1 |
| (0.930-0.969) | (80.9-95.9) | (81.7-97.2) | (86.6-92.1) | (85.5-97.0) | (81.3-94.7) |
| MF 0.892 | 79.6 | 86.7 | 81.9 | 93.3 | 64.6 |
| (0.860-0.924) | (71.8-92.2) | (72.6-94.1) | (77.1-87.4) | (88.6-96.7) | (57.5-80.2) |

External testing 1 at Prince of Wales Hospital (PWH), Hong Kong SAR, China

| | | | | | |
|---|---|---|---|---|---|
| GON 0.890 | 78.9 | 86.1 | 82.0 | 86.9 | 77.7 |
| (0.864-0.917) | (70.4-86.4) | (77.3-92.8) | (78.7-85.1) | (81.4-92.4) | (72.1-83.3) |
| MF 0.885 | 83.8 | 81.5 | 83.1 | 88.2 | 75.5 |
| (0.855-0.915) | (74.4-93.8) | (69.3-90.2) | (79.1-86.6) | (83.2-92.9) | (67.3-87.5) |

External testing 2 at Tuen Mun Eye Centre (TMEC), Hong Kong SAR, China

| | | | | | |
|---|---|---|---|---|---|
| GON 0.903 | 77.6 | 91.9 | 84.3 | 92.1 | 78.4 |
| (0.867-0.939) | (67.1-86.7) | (83.1-98.4) | (80.2-88.4) | (85.0-98.2) | (72.0-85.4) |
| MF 0.855 | 83.7 | 76.5 | 79.8 | 78.3 | 81.8 |
| (0.811-0.899) | (68.9-91.9) | (66.7-87.9) | (74.9-84.6) | (72.5-86.5) | (72.9-89.5) |

External testing 3 at Alice Ho Miu Ling Nethersole Hospital (AHNH), Hong Kong SAR, China

| | | | | | |
|---|---|---|---|---|---|
| GON 0.906 | 79.7 | 88.9 | 82.1 | 94.4 | 64.9 |
| (0.880-0.933) | (68.5-88.1) | (79.1-96.7) | (76.5-86.6) | (90.5-98.2) | (56.2-74.7) |
| MF 0.886 | 78.3 | 88.1 | 80.6 | 95.6 | 54.7 |
| (0.856-0.916) | (72.8-84.6) | (79.7-94.1) | (76.3-84.7) | (93.0-97.8) | (49.1-61.9) |

External testing 4 at Byers Eye Institute, Stanford University (Stanford), the United States

| | | | | | |
|---|---|---|---|---|---|
| GON 0.950 | 85.2 | 94.0 | 87.3 | 97.9 | 65.6 |
| (0.936-0.963) | (79.0-92.5) | (86.5-98.1) | (83.2-91.1) | (95.8-99.3) | (58.1-77.4) |
| MF 0.866 | 68.4 | 95.0 | 79.5 | 94.7 | 69.1 |
| (0.843-0.888) | (62.9-77.0) | (86.2-97.7) | (77.1-82.0) | (88.0-97.6) | (66.0-74.0) |

External testing 5 at Singapore Eye Research Institute (SERI), Singapore

| | | | | | |
|---|---|---|---|---|---|
| GON 0.930 | 83.9 | 92.2 | 88.2 | 90.9 | 86.1 |
| (0.915-0.946) | (80.4-87.2) | (89.6-94.7) | (86.3-90.0) | (88.2-93.6) | (83.6-88.6) |
| MF 0.875 | 84.1 | 76.5 | 79.8 | 73.2 | 86.2 |
| (0.854-0.896) | (70.7-90.2) | (69.3-88.3) | (77.0-82.2) | (68.7-82.4) | (79.6-90.4) |

In creating certain embodiments, the inventors utilized a series of data pre-processing methods and on-the-fly data augmentation methods to train the provided DL model with reduced GPU memory costs while avoiding the over-fitting issue. Embodiments provide 3D DL models to analyze the OCT volumetric images. Embodiments provide a multi-task technique to develop a 3D DL model for classifying both GON and MF.

Embodiments have withstood external testing from different centers in different countries featuring different patient populations for the provided DL models, verifying generalizability as shown in Table 1 and Table 2.

Embodiments provide class activation maps to visualize the discriminative features (i.e., heatmaps). The feature maps, i.e., the intermediate outputs of the network layers, before the global average pooling layer as well as the parameters of the fully connected layer can be used to obtain the heatmap. In one exemplary embodiment there are 256 feature maps each with dimension 4×4×32, while the parameters of the fully connected layer are of dimension 1×1×1×256. The sum of the feature maps weighted by the parameters can be taken to generate the class activation map. Each entry in the weights represents the importance of each feature map. Finally, the class activation map can be resized to the same dimension of the original OCT image by interpolation to obtain the heatmap. These heatmaps can provide end-users some insights on where the discriminative areas are for the AI to detect diseases.

Embodiments provide an AI-platform that integrates the provided image quality control DL model into the provided disease classification model, which provides a more accurate disease detection.

Embodiments of the provided AI-platform can be implemented into available commercialized OCT devices and configured to automatedly detect the exported data for further image analysis and disease detection.

Embodiments of the provided AI-platform can also integrate other DL models for other disease detection.

Embodiments of the subject invention address the technical problem of detecting GON and MF from imaging such as OCT image data being expensive, needing excessive human processing and experience in ophthalmology, not being suitable for rapid screening, and requiring expert resources to complete.

This problem is addressed by providing a system for 3D AI-aided classification using digital image processing in which a machine learning method applying a combination of advanced techniques is utilized within an AI-platform to provide easy-to-interpret AI reports with outputs including image quality, glaucoma classification, myopic features classification, AI scores, and referral suggestions within a few minutes.

The transitional term “comprising,” “comprises,” or “comprise” is inclusive or open-ended and does not exclude additional, unrecited elements or method steps. By contrast, the transitional phrase “consisting of” excludes any element, step, or ingredient not specified in the claim. The phrases “consisting of” or “consists essentially of” indicate that the claim encompasses embodiments containing the specified materials or steps and those that do not materially affect the basic and novel characteristic(s) of the claim. Use of the term “comprising” contemplates other embodiments that “consist of” or “consisting essentially of” the recited component(s).

When ranges are used herein, such as for dose ranges, combinations and subcombinations of ranges (e.g., subranges within the disclosed range), specific embodiments therein are intended to be explicitly included. When the term “about” is used herein, in conjunction with a numerical value, it is understood that the value can be in a range of 95% of the value to 105% of the value, i.e., the value can be +/−5% of the stated value. For example, “about 1 kg” means from 0.95 kg to 1.05 kg.

The methods and processes described herein can be embodied as code and/or data. The software code and data described herein can be stored on one or more machine-readable media (e.g., computer-readable media), which may include any device or medium that can store code and/or data for use by a computer system. When a computer system and/or processor reads and executes the code and/or data stored on a computer-readable medium, the computer system and/or processor performs the methods and processes embodied as data structures and code stored within the computer-readable storage medium.

It should be appreciated by those skilled in the art that computer-readable media include removable and non-removable structures/devices that can be used for storage of information, such as computer-readable instructions, data structures, program modules, and other data used by a computing system/environment. A computer-readable medium includes, but is not limited to, volatile memory such as random access memories (RAM, DRAM, SRAM); and non-volatile memory such as flash memory, various read-only-memories (ROM, PROM, EPROM, EEPROM), magnetic and ferromagnetic/ferroelectric memories (MRAM, FeRAM), and magnetic and optical storage devices (hard drives, magnetic tape, CDs, DVDs); network devices; or other media now known or later developed that are capable of storing computer-readable information/data. Computer-readable media should not be construed or interpreted to include any propagating signals. A computer-readable medium of embodiments of the subject invention can be, for example, a compact disc (CD), digital video disc (DVD), flash memory device, volatile memory, or a hard disk drive (HDD), such as an external HDD or the HDD of a computing device, though embodiments are not limited thereto. A computing device can be, for example, a laptop computer, desktop computer, server, cell phone, or tablet, though embodiments are not limited thereto.

The invention may be better understood by reference to certain illustrative exemplary embodiments, including but not limited to the following:

Embodiment 1. A system for three-dimensional (3D) artificial intelligence (AI)-aided classification of glaucomatous optic neuropathy (GON) and myopic optic disc morphology (MF) from optical coherence tomography (OCT) scans, the system comprising:
a user interface subsystem;
an information management subsystem; and
an artificial intelligence (AI) image analysis subsystem comprising:
   a pre-diagnosis image quality assessment model, and
   a GON and MF multi-task detection model.

Embodiment 2. The system of Embodiment 1, wherein the pre-diagnosis image quality assessment model comprises a three-dimensional squeeze-and-excitation (SE) model.

Embodiment 3. The system of Embodiment 2, wherein the three-dimensional SE model is based on a ResNeXt architecture.

Embodiment 4. The system of Embodiment 3, wherein the three-dimensional SE model comprises a multiplicity of SE-ResNeXt building blocks.

Embodiment 5. The system of Embodiment 4, wherein the three-dimensional SE model comprises batch normalization, global average pooling, convolution, and averaging.

Embodiment 6. The system of Embodiment 1, wherein the 3D SE model is configured and adapted to produce an output comprising a confirmation of image quality sufficient for reliable operation of the GON and MF multi-task detection model.

Embodiment 7. The system of Embodiment 6, wherein the GON and MF multi-task detection model comprises a shared feature extraction module configured and adapted to deliver a respective input to each of a GON classification module and an MF detection module.

Embodiment 8. The system of Embodiment 7, wherein the shared feature extraction module comprises convolution, batch normalization, a multiplicity of residual units, and global average pooling.

Embodiment 9. The system of Embodiment 7, wherein the GON classification module, and the MF detection module each, respectively, comprise a fully connected softmax layer.

Embodiment 10. The system of Embodiment 7, wherein the GON and MF multi-task detection model utilizes ResNet-37 in a 3D version.

Embodiment 11. The system of Embodiment 7, wherein the pre-diagnosis image quality assessment model comprises an irrelevancy reduction mechanism and a self-attention mechanism; and the GON and MF multi-task detection model is trained using a multi-task learning paradigm.

Embodiment 12. An artificial intelligence (AI) enhanced system for three-dimensional classification of glaucomatous optic neuropathy (GON) and myopic optic disc morphology (MF) from optical coherence tomography (OCT) scans, the system comprising:
an OCT scanner;
a processor in operable communication with the OCT scanner; and
a machine-readable medium in operable communication with the processor and having instructions stored thereon that, when executed by the processor, perform the following steps:
   a) obtaining, from the OCT scanner, a three-dimensional OCT image dataset representing an eye of a patient;
   b) processing the three-dimensional OCT image dataset through a pre-diagnosis image quality assessment model to produce an image quality assessment rating;
   c) comparing the image quality assessment rating against a first predetermined value to confirm sufficient image quality of the three-dimensional OCT image dataset for further processing within the system;
   d) processing the three-dimensional OCT image dataset through a GON and MF multi-task detection model to produce an AI-GON-score and an AI-MF-score for the three-dimensional OCT image dataset;
   e) comparing the AI-GON-score against a second predetermined value to produce an AI-GON-analysis-result; and
   f) comparing the AI-MF-score against a third predetermined value to produce an AI-MF-analysis-result.

Embodiment 13. The system according to Embodiment 1, wherein:
the three-dimensional OCT image dataset representing an eye of the patient is a first three-dimensional OCT image dataset representing a right eye of the patient;
the image quality assessment rating is a first image quality assessment rating;
the AI-GON-score is a first AI-GON-score;
the AI-MF-score is a first AI-MF-score;
the AI-GON-analysis-result is a first AI-GON-analysis-result;
the AI-MF-analysis-result is a first AI-MF-analysis-result; and
wherein the instructions when executed further repeat steps a)-f) with respect to a second three-dimensional OCT image dataset representing a left eye of the patient, thus producing a second image quality assessment rating, a second AI-GON-score, a second AI-MF-score, a second AI-GON-analysis-result, and a second AI-MF-analysis-result.

Embodiment 14. The system according to Embodiment 12, the instructions when executed further performing the following additional steps:
g) reporting the image quality assessment rating, the AI-GON-score, the AI-MF-score, the AI-GON-analysis-result, and the AI-MF-analysis-result.

Embodiment 15. The system according to Embodiment 13, the instructions when executed further performing the following additional steps:
g) reporting the first image quality assessment rating, the first AI-GON-score, the first AI-MF-score, the first AI-GON-analysis-result, and the first AI-MF-analysis-result; and
h) reporting the second image quality assessment rating, the second AI-GON-score, the second AI-MF-score, the second AI-GON-analysis-result, and the second AI-MF-analysis-result.

Embodiment 16. The system according to Embodiment 15, the instructions when executed further performing the following additional steps:
i) producing a referral-triage suggestion based on any combination of the first image quality assessment rating, the first AI-GON-score, the first AI-MF-score, the first AI-GON-analysis-result, the first AI-MF-analysis-result, the second image quality assessment rating, the second AI-GON-score, the second AI-MF-score, the second AI-GON-analysis-result, and the second AI-MF-analysis-result, respectively.

Embodiment 17. The system according to Embodiment 16, the instructions when executed further performing the following additional steps:
j) producing a clinical management suggestion based on the referral-triage suggestion.

Embodiment 18. The system according to Embodiment 17, the instructions when executed further performing the following additional steps:
k) comparing one of the image quality assessment rating, the first image quality assessment rating, or the second image quality assessment rating against the first predetermined value and failing to confirm sufficient image quality of the respective three-dimensional OCT image dataset for further processing within the system, thus producing an ungradable image dataset;
l) obtaining, from the OCT scanner, a replacement three-dimensional OCT image dataset;
m) replacing the ungradable image dataset with the replacement three-dimensional OCT image dataset; and
n) repeating steps b)-f) with respect to the replacement three-dimensional OCT image dataset.

Embodiment 19. A system for rapid three-dimensional artificial intelligence-aided classification of glaucomatous optic neuropathy (GON) and myopic optic disc morphology (MF) from optical coherence tomography (OCT) scans, the system comprising:
a user interface subsystem;
an information management subsystem; and
an artificial intelligence (AI) image analysis subsystem comprising:
   a pre-diagnosis image quality assessment model, and
   a GON and MF multi-task detection model;
wherein the pre-diagnosis image quality assessment model comprises a 3D squeeze-and-excitation (SE) model based on a ResNeXt architecture and comprising a multiplicity of SE-ResNeXt building blocks;
wherein the 3D SE model comprises batch normalization, global average pooling, convolution, and averaging;
wherein the 3D SE model is configured and adapted to produce an output comprising a confirmation of image quality sufficient for reliable operation of the GON and MF multi-task detection model; and
wherein the GON and MF multi-task detection model comprises a shared feature extraction module configured and adapted to deliver a respective input to each of a GON classification module and an MF detection module.

Embodiment 20. The system of Embodiment 19, wherein the shared feature extraction module comprises convolution, batch normalization, a multiplicity of residual units, and global average pooling;
wherein the GON classification module, and the MF detection module each, respectively, comprise a fully connected softmax layer;
wherein the GON and MF multi-task detection model utilizes ResNet-37 in a 3D version;
wherein the pre-diagnosis image quality assessment model comprises an irrelevancy reduction mechanism and a self-attention mechanism; and the GON and MF multi-task detection model is trained using a multi-task learning paradigm.

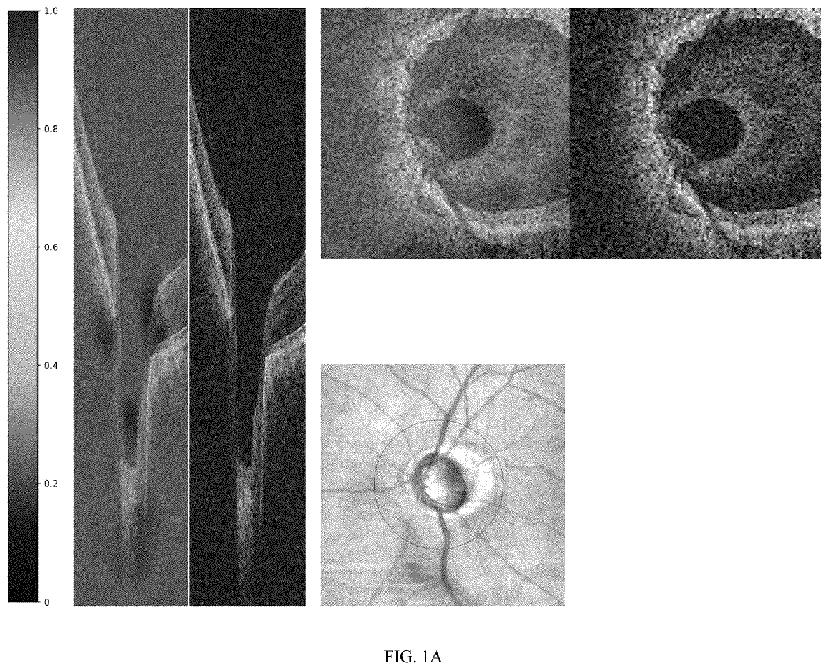

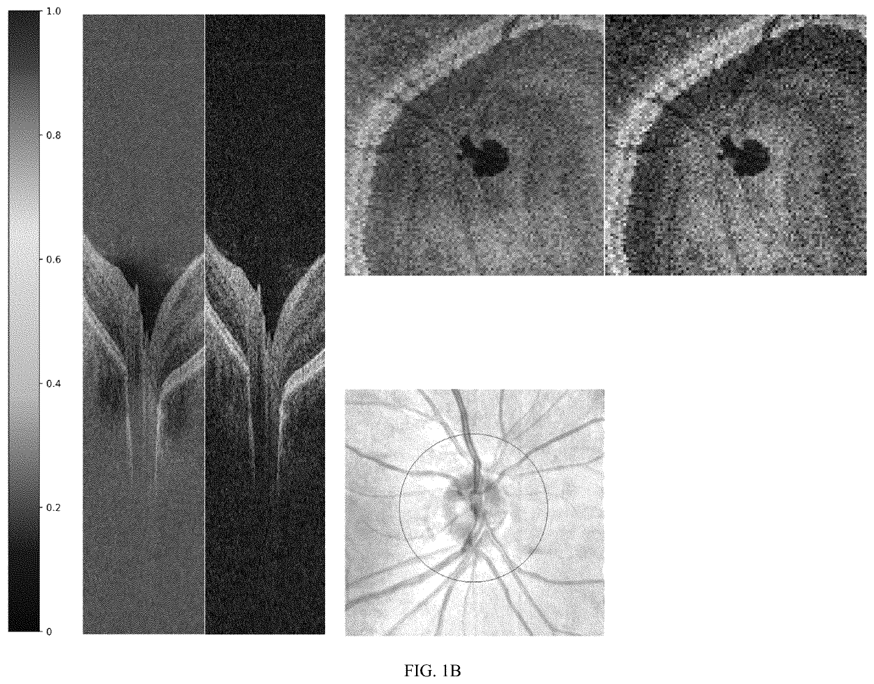

Turning now to the figures, FIGS. 1A-1B show examples of correctly detected eyes with glaucoma and eyes without glaucoma detected by a 3D DL model according to an embodiment of the subject invention, the corresponding heatmaps generated by Class Activation Map (CAM), and the paired en face fundus images. The red-orange colored area on heatmaps has the most discriminatory power to detect glaucoma. The green-blue colored area on heatmaps showed no abnormalities. (1A) A correctly detected OCT volumetric scan as “yes glaucoma” from the cross-sectional (left side images) and top (top-right images) view as well as the paired enface 2D fundus (bottom-central image.) The heatmaps showed that, in addition to the common glaucomatous structural damage areas, such as the RNFL and neuroretinal rim, other areas covering the lamina cribrosa (LC) and choroid can be related to the detection of glaucoma by the 3D DL model. (1B) A correctly detected OCT volumetric scan as “no glaucoma” from the cross-sectional (left side images) and top (top-right images) view as well as the corresponding enface 2D fundus (bottom-central image.) The heatmaps showed that the majority of the pixels in the images were blue-green colored.

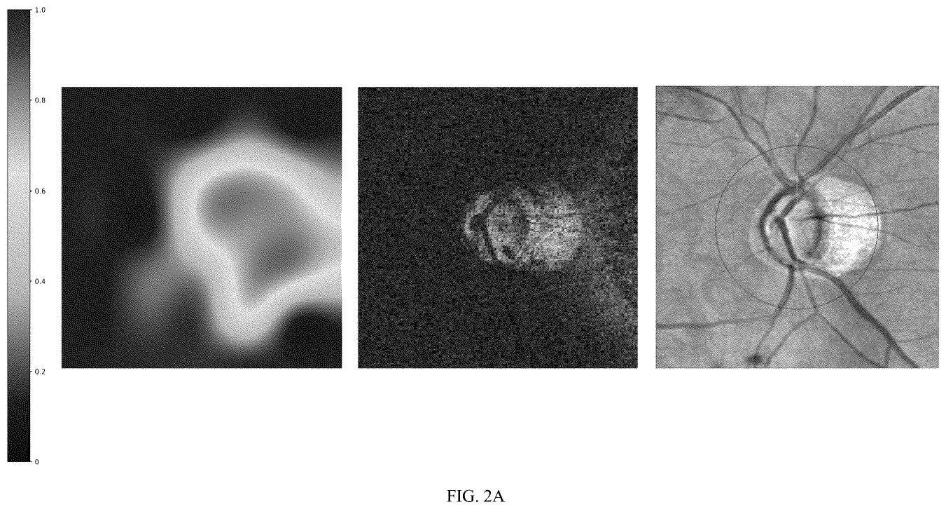

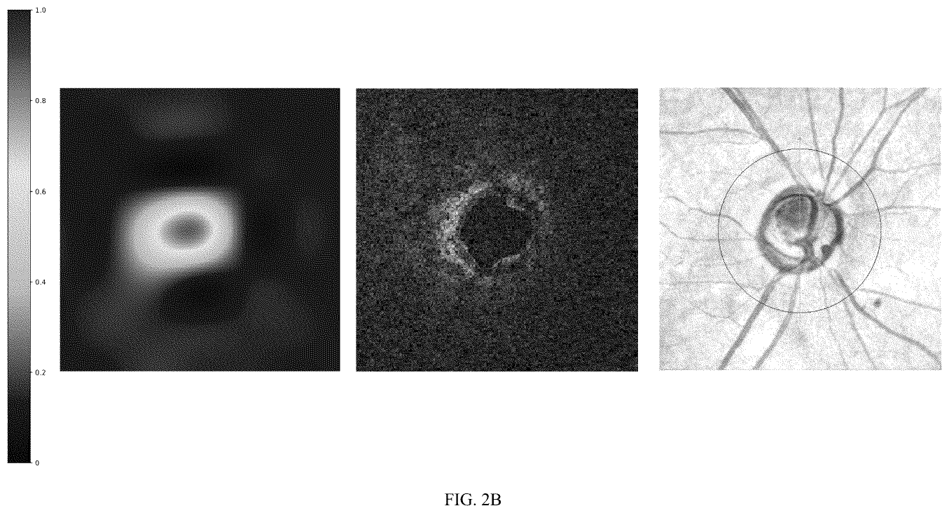

FIGS. 2A-2B show examples of truly detected eyes by a multi-task 3D DL model according to an embodiment of the subject invention. From left to right were heatmaps, raw images, and the corresponding enface fundus images. The red-orange colored area on the respective heatmaps has the most discriminatory power to detect myopia structural changes. The heatmaps shows in (2A) an eye with myopic features (e.g., peripapillary atrophy) the optic disc area and the areas with PPA is red-orange colored. In (2B) is shown an eye without myopic features, only the optic disc was red-orange colored.

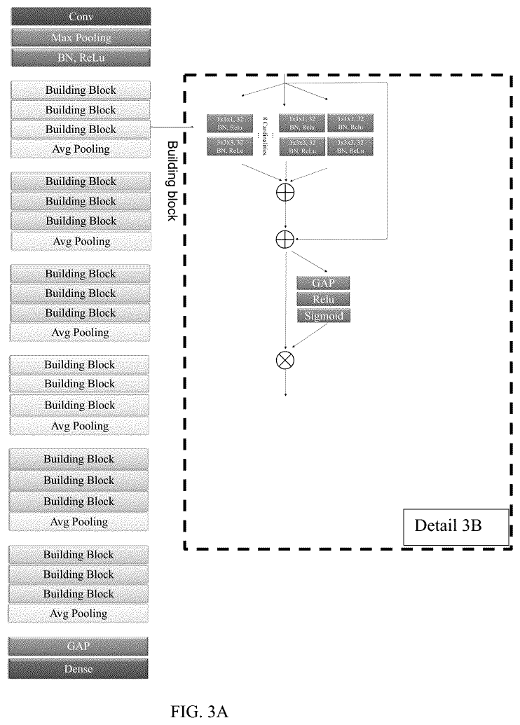

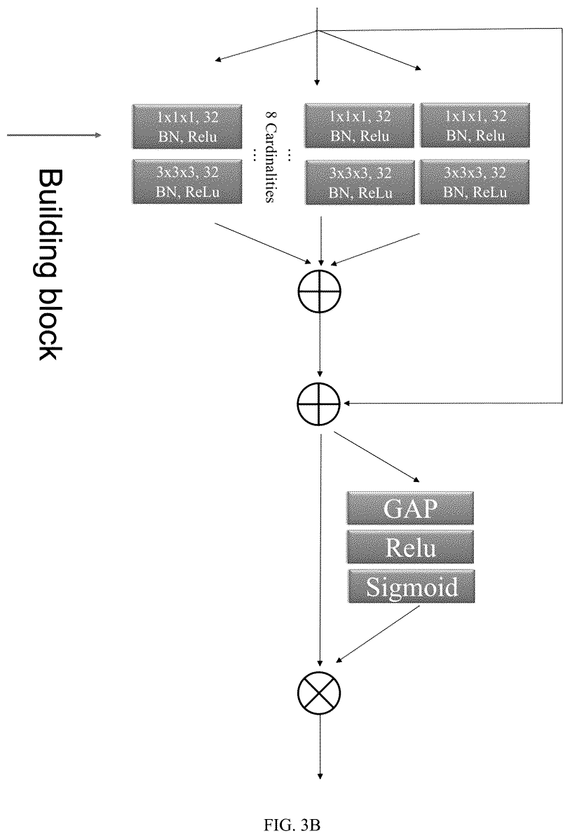

FIGS. 3A-3B show image quality assessment using a squeeze-and-excitation (SE)-ResNeXt model in 3D version according to an embodiment of the subject invention. In each SE-ResNeXt block, the SE reduction ratio was set to 4 and the cardinalities of the transformation layer were set to 8, with 32 filters. These diagrams illustrate the architecture of basic building blocks and the architecture of different models. (3A) The architecture of the model with SE-ResNeXt building blocks. (3B) The details of SE-ResNeXt building blocks. The inventors used eight transformation layers along with 32 filters for each transformation layer. SE=squeeze-and-excitation. BN=batch normalization, GAP=global average pooling, Conv=convolutional, Avg=average.

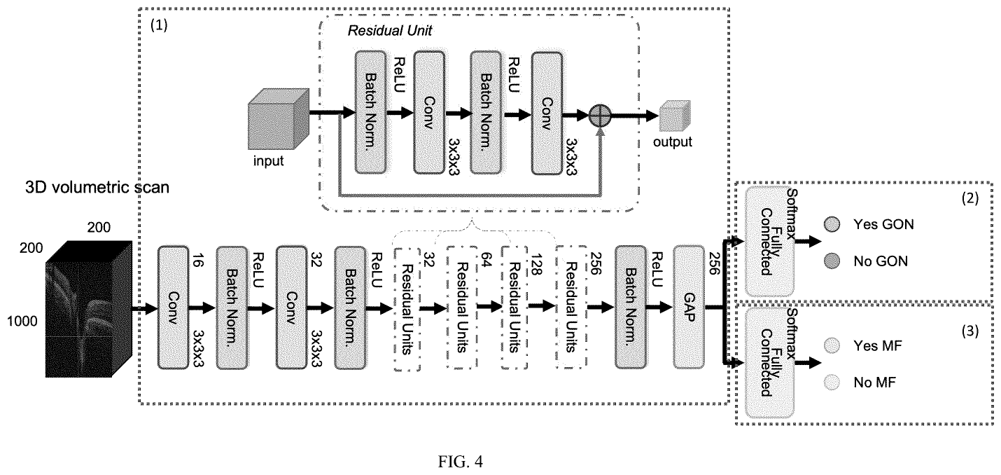

FIG. 4 is a diagram showing the structure of a 3D multi-task deep learning model according to an embodiment of the subject invention. For the GON and MF multi-task model, certain embodiments utilize ResNet-37 in a 3D version. One embodiment of the provided network includes three modules (e.g., as shown in FIG. 4): (1) shared feature extraction module, (2) GON classification module, and (3) MF detection module, respectively. This network was built based on a ResNet-37 network with 3D convolutional layers and a global average pooling layer. The input was an OCT volumetric scan of size 200×1000×200 pixels after image pre-processing and the output was Yes/No GON and Yes/No ME.

FIG. 5 is a diagram showing the user interface for image quality assessment, GON and MF detection according to an embodiment of the subject invention. Embodiments provide an information management system and a user interface integrated with AI models. The provided AI system can be incorporated into commercially available devices (e.g., Cirrus OCT device) as a built-in or standalone software, system, or module.

FIGS. 6A-6B show the workflow and an AI report generated by the intervention (i.e., the 3D AI-aided classification systems for GON and MF from OCT scans, which is an AI platform integrating an information management system, an AI image analysis system, and a user interface) according to an embodiment of the subject invention. By way of example, but not limitation, the image quality can first be assessed by the AI system and the AI output can be sufficient or insufficient. The end-user can either retake ungradable scans or click by-pass function for images with AI output of insufficient to conduct further disease detection. By way of example, but not limitation, an AI score larger than 55% can be Yes GON or Yes MF, respectively; less than 45% can be No GON or NO MF, respectively; between 45%-55% can be assigned as an uncertain case.

By way of example, but not limitation, referral suggestions can include: 1) “Refer to glaucoma specialists”: any eye “Yes GON” with or without any eye “Yes MF”; 2) “Non urgent referral”: both eyes “No GON” and any eye “Yes MF”; 3) “Observe only”: both eyes “No GON” and both eyes “No MF”.

The benefits of the intervention by providing a clear AI report with outcomes of image quality (e.g., a binary determination such as, “sufficient” or “insufficient”), GON detection (e.g., a binary determination such as, “Yes GON” or “No GON”), MF detection (e.g., a binary determination such as, “Yes MF” or “No MF”) with AI scores, and referral suggestions will be more user-friendly and efficient for end-users' interpretation in a busy clinic. If there are further needs, end-users can also review the raw images via the information management system.

### Materials and Methods

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1 Creation of an Image Quality Control Deep Learning Model

Data augmentation strategies, including random flipping, random rotating, and random shifting, were used to enhance the training samples and alleviate overfitting. The original OCT volumes were with size of 200×200×1024 in three axes, x-axis, y-axis, and z-axis, respectively. To mimic the real OCT imaging in clinical practice, some data augmentation methods were only applied on one or two axes for the whole volume. For instance, 20% chance random flipping, 15-degree random rotation were applied on only x-axis (200) and y-axis (200). The color channel was set to one since all OCT images were grey scaled.

The DL model was implemented with Keras and Tensorflow, on a workstation equipped with i9-7900× and Nvidia GeForce GTX 1080Ti. Firstly, there were 32 filters with 7×7×7 kernel convolution layer with the stride of 2, along with a 3×3×3 max pooling with the same stride setting. Secondly, the obtained feature maps went through 18 ResNet blocks. A pooling size 2 with stride 2 average pooling was performed every 3 blocks to aggregate the learnt features. Channel-wise batch normalization and ReLU activation were performed after all convolution operations. Finally, a global average pooling followed by a fully connected softmax layer was used to produce the binary output as gradable or ungradable. This ResNet-based model was taken as the benchmark model. The inventors further experimented with the SE-ResNet-block²⁷ and SE-ResNeXt-block²⁸, as the basic building block. In each SE-ResNet or SE-ResNeXt block, the SE reduction ratio was set to 4 and the cardinalities of the transformation layer were set to 8, with 32 filters. Cross-entropy and Adam were used as the loss function and the optimizer. During the training, 3,000 volumetric scans were selected with data balancing. Batch size was set to one due to the limited GPU memory. The initial learning rate was set to 0.0001, and then reduced by multiplying 0.75 in every 2 epochs.

### Example 2 Creation of a Multi-Task DL Model for Glaucoma and Myopic Structure Changes Classification

The inventors applied standardization and normalization for data pre-processing. Specifically, standardization was used to transfer data to have zero mean and unit variance, and normalization rescaled the data to the range of 0 to 1. To alleviate the over-fitting issue, during the training process, the inventors used several data augmentation techniques, including random cropping and random flipping at three axes, to enrich training samples for the 3D OCT volumetric data. Consequently, the final input size of the network was 200×1000×200. The inventors implemented the DL model using Keras package and python on a workstation equipped with 3.5 GHz Intel® Core™ i7-5930K CPU and GPUs of Nvidia GeForce GTX Titan X. The inventors set the learning rate as 0.0001 and optimized the weights of the networks with Adam stochastic gradient descent algorithm.

The provided network included three modules, 1) shared feature extraction module, 2) glaucoma classification module, and 3) myopic features detection module, respectively. The constructed network was similar to the inventors' previous study²⁹ with ResNet-37 as the backbone. The inventors used shortcut connections to perform identity mapping and evade the vanishing gradient problem during backpropagation. The inventors removed the fully connected layer from the 3D ResNet-37. This module acted as the shared feature extraction module. In the GON classification module, a fully connected layer with softmax activation accepted the feature from the first module and output the classification probabilities for “Yes GON” and “No GON”. Likewise, there was also a fully connected layer with softmax activation in the MF detection module and output the classification probabilities for “Yes MF” and “No MF”.

All gradable OCT volumetric scans were randomly divided for training (80%), tuning (10%), and internal validation (10%) at the patient level. In each set, the ratio of “Yes GON & Yes MF”, “Yes GON & No MF”, “No GON & Yes MF”, and “No GON & No MF” was similar, and multiple images from the same subjects were in the same set to inhibit leakage or performance overestimation. The inventors trained the multi-task DL model from scratch, and the tuning dataset was used to select and modify the optimum model during training. During the training, tuning, and internal validation, the inventors observed the training-validation curve to evaluate for any over-fitting issue, which could also provide a further reference to the generalizability of the models. Additionally, OCT volumetric scans from 5 centers were used for external testing.

Finally, the inventors generated heatmaps for selected eyes by class activation map (CAM)³⁰ to visualize the classification. Results are seen in Table 1, Table 2, and FIGS. 1A, 1, 2A, and 2B.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Bourne R R, Stevens G A, White R A, et al. Causes of vision loss worldwide, 1990-2010: a systematic analysis. Lancet Glob Health 2013; 1(6): e339-49.
2. Tham Y C, Li X, Wong TY, Quigley H A, Aung T, Cheng C Y. Global prevalence of glaucoma and projections of glaucoma burden through 2040: a systematic review and meta-analysis. Ophthalmology 2014; 121(11): 2081-90.
3. Leung C K, Cheung C Y, Weinreb R N, et al. Retinal nerve fiber layer imaging with spectral-domain optical coherence tomography: a variability and diagnostic performance study.

Ophthalmology 2009; 116(7): 1257-63, 63 el-2.
4. Koh V, Tham Y C, Cheung C Y, et al. Diagnostic accuracy of macular ganglion cell-inner plexiform layer thickness for glaucoma detection in a population-based study: Comparison with optic nerve head imaging parameters. Plos One 2018; 13(6): e0199134.
5. Mwanza J C, Budenz D L, Godfrey D G, et al. Diagnostic performance of optical coherence tomography ganglion cell-inner plexiform layer thickness measurements in early glaucoma. Ophthalmology 2014; 121(4): 849-54.
6. Chang R T, Knight O J, Feuer W J, Budenz D L. Sensitivity and specificity of time-domain versus spectral-domain optical coherence tomography in diagnosing early to moderate glaucoma. Ophthalmology 2009; 116(12): 2294-9.
7. Hardin J S, Taibbi G, Nelson S C, Chao D, Vizzeri G. Factors Affecting Cirrus-HD OCT Optic Disc Scan Quality: A Review with Case Examples. J Ophthalmol 2015; 2015: 746150.
8. Chhablani J, Krishnan T, Sethi V, Kozak I. Artifacts in optical coherence tomography. Saudi J Ophthalmol 2014; 28(2): 81-7.
9. Liu S, Paranjape A S, Elmaanaoui B, et al. Quality assessment for spectral domain optical coherence tomography (OCT) images. Proc SPIE Int Soc Opt Eng 2009; 7171: 71710X.
10. Lee R, Tham Y C, Cheung C Y, et al. Factors affecting signal strength in spectral-domain optical coherence tomography. Acta Ophthalmol 2018; 96(1): e54-e8.
11. Cheung C Y L, Leung C K S, Lin D S, Pang C P, Lam D S C. Relationship between retinal nerve fiber layer measurement and signal strength in optical coherence tomography. Ophthalmology 2008; 115(8): 1347-51.
12. Cheung C Y, Chan N, Leung C K. Retinal Nerve Fiber Layer Imaging with Spectral-Domain Optical Coherence Tomography: Impact of Signal Strength on Analysis of the RNFL Map. Asia Pac J Ophthalmol (Phila) 2012; 1(1): 19-23.
13. Baniasadi N, Wang M Y, Wang H, Mahd M, Elze T. Associations between Optic Nerve Head-Related Anatomical Parameters and Refractive Error over the Full Range of Glaucoma Severity. Transl Vis Sci Techn 2017; 6(4).
14. Yan Y N, Wang Y X, Xu L, Xu J, Wei W B, Jonas J B. Fundus Tessellation: Prevalence and Associated Factors: The Beijing Eye Study 2011. Ophthalmology 2015; 122(9): 1873-80.
15. Hwang Y H, Yoo C, Kim Y Y. Myopic optic disc tilt and the characteristics of peripapillary retinal nerve fiber layer thickness measured by spectral-domain optical coherence tomography. J Glaucoma 2012; 21(4): 260-5.
16. Jonas J B, Jonas S B, Jonas R A, et al. Parapapillary atrophy: histological gamma zone and delta zone. Plos One 2012; 7(10): e47237.
17. Qiu K L, Zhang M Z, Leung C K S, et al. Diagnostic Classification of Retinal Nerve Fiber Layer Measurement in Myopic Eyes: A Comparison Between Time-Domain and Spectral-Domain Optical Coherence Tomography. American Journal of Ophthalmology 2011; 152(4): 646-53.
18. LeCun Y, Bengio Y, Hinton G. Deep learning. Nature 2015; 521(7553): 436-44.
19. Xiangyu C, Yanwu X, Damon Wing Kee W, Tien Yin W, Jiang L. Glaucoma detection based on deep convolutional neural network. Conf Proc IEEE Eng Med Biol Soc 2015; 2015: 715-8.
20. Muhammad H, Fuchs T J, De Cuir N, et al. Hybrid Deep Learning on Single Wide-field Optical Coherence tomography Scans Accurately Classifies Glaucoma Suspects. J Glaucoma 2017; 26(12): 1086-94.
21. Christopher M, Belghith A, Bowd C, et al. Performance of Deep Learning Architectures and Transfer Learning for Detecting Glaucomatous Optic Neuropathy in Fundus Photographs. Sci Rep 2018; 8(1): 16685.
22. Medeiros F A, Jammal A A, Thompson A C. From Machine to Machine: An OCT-Trained Deep Learning Algorithm for Objective Quantification of Glaucomatous Damage in Fundus Photographs. Ophthalmology 2018.
23. Maetschke S, Antony B, Ishikawa H, Wollstein G, Schuman J, Garnavi R. A feature agnostic approach for glaucoma detection in OCT volumes. Plos One 2019; 14(7).
24. Thompson A C, Jammal A A, Medeiros F A. A Deep Learning Algorithm to Quantify Neuroretinal Rim Loss from Optic Disc Photographs. Am J Ophthalmol 2019.
25. Li Z, He Y, Keel S, Meng W, Chang R T, He M. Efficacy of a Deep Learning System for Detecting Glaucomatous Optic Neuropathy Based on Color Fundus Photographs. Ophthalmology 2018; 125(8): 1199-206.
26. Shibata N, Tanito M, Mitsuhashi K, et al. Development of a deep residual learning algorithm to screen for glaucoma from fundus photography. Sci Rep 2018; 8(1): 14665.
27. Jie Hu L S, Samuel Albanie, Gang Sun, Enhua Wu. Squeeze-and-Excitation Networks. arXiv:170901507 2018.
28. Saining Xie R G, Piotr Dolla'r, Zhuowen Tu, Kaiming He. Aggregated Residual Transformations for Deep Neural Networks. arXiv preprint arXiv: 161105431 2016.
29. Ran A R, Cheung C Y, Wang X, et al. Detection of glaucomatous optic neuropathy with spectral-domain optical coherence tomography: a retrospective training and validation deep-learning analysis. The Lancet Digital Health 2019; 1(4): e172-e82.
30. Zhou B, Khosla A, Lapedriza A, Oliva A, Torralba A. Learning Deep Features for Discriminative Localization. Proc Cvpr Ieee 2016: 2921-9.
31. Ran A R, Cheung C Y, Wang X, Chen H, Luo LY, Chan P P, Wong M O M, Chang R T, Mannil S S, Young A L, Pang C P, Heng P A, Tham C C. Detection of glaucomatous optic neuropathy with spectral-domain optical coherence tomography: a retrospective training and validation deep-learning analysis. The Lancet Digital Health. 2019;1(4):e172-e82.
32. Ran A R, Shi J, Ngai A K, Chan W Y, Chan P P, Young A L, Yung H W, Tham C C, Cheung C Y. Artificial intelligence deep learning algorithm for discriminating ungradable optical coherence tomography three-dimensional volumetric optic disc scans. Neurophotonics. 2019 Oct;6(4):041110.
33. Ran A R, Wang X, Chan P P, Chan N C, Yip W, Young A L, Wong M O M, Yung H W, Chang R T, Mannil S S, Tham Y C, Cheng C Y, Chen H, Li F, Zhang X, Heng P A, Tham C C, Cheung CY. Three-Dimensional Multi-Task Deep Learning Model to Detect Glaucomatous Optic Neuropathy and Myopic Features From Optical Coherence Tomography Scans: A Retrospective Multi-Centre Study. Front Med. 2022 Jun. 15; 9:860574.
34. Wang X, Chen H, Ran A R, Luo L Y, Chan P P, Tham C C, Chang R T, Mannil S S, Cheung C Y, Heng P A. Towards multi-center glaucoma OCT image screening with semi-supervised joint structure and function multi-task learning. Medical image Analysis. 2020 May; 19(63):101695. (DOI:10.1016/j.media.2020.101695)
35. Ran A R, Tham C C, Chan P P, Cheng C Y, Tham Y C, Rim T H, Cheung C Y. Deep learning in glaucoma with optic coherence tomography: a review. Eye. 2020 Oct:1-14.

We claim:

1. A system for rapid three-dimensional (3D) artificial intelligence (AI)-aided classification of glaucomatous optic neuropathy (GON) and myopic optic disc morphology (MF) from optical coherence tomography (OCT) scans, the system comprising:
a user interface subsystem;
an information management subsystem; and
an AI image analysis subsystem comprising:
a pre-diagnosis image quality assessment model, and
a GON and MF multi-task detection model.

2. The system of claim 1, wherein the pre-diagnosis image quality assessment model comprises a three-dimensional squeeze-and-excitation (SE) model.

3. The system of claim 2, wherein the three-dimensional SE model is based on a ResNeXt architecture.

4. The system of claim 3, wherein the three-dimensional SE model comprises a multiplicity of SE-ResNeXt building blocks.

5. The system of claim 4, wherein the three-dimensional SE model comprises batch normalization, global average pooling, convolution, and averaging.

6. The system of claim 1, wherein the three-dimensional SE model is configured and adapted to produce an output comprising a confirmation of image quality sufficient for reliable operation of the GON and MF multi-task detection model.

7. The system of claim 6, wherein the GON and MF multi-task detection model comprises a shared feature extraction module configured and adapted to deliver a respective input to each of a GON classification module and an MF detection module.

8. The system of claim 7, wherein the shared feature extraction module comprises convolution, batch normalization, a multiplicity of residual units, and global average pooling.

9. The system of claim 7, wherein the GON classification module, and the MF detection module each, respectively, comprises a fully connected softmax layer.

10. The system of claim 7, wherein the GON and MF multi-task detection model utilizes ResNet-37 in a 3D version.

11. The system of claim 7, wherein the pre-diagnosis image quality assessment model comprises an irrelevancy reduction mechanism and a self-attention mechanism; and the GON and MF multi-task detection model is trained using a multi-task learning paradigm.

12. An artificial intelligence (AI) enhanced system for rapid three-dimensional classification of glaucomatous optic neuropathy (GON) and myopic optic disc morphology (MF) from optical coherence tomography (OCT) scans, the system comprising:
an OCT scanner;
a processor in operable communication with the OCT scanner; and
a machine-readable medium in operable communication with the processor and having instructions stored thereon that, when executed by the processor, perform the following steps:
a) obtaining, from the OCT scanner, a three-dimensional OCT image dataset representing an eye of a patient;
b) processing the three-dimensional OCT image dataset through a pre-diagnosis image quality assessment model to produce an image quality assessment rating;
c) comparing the image quality assessment rating against a first predetermined value to confirm sufficient image quality of the three-dimensional OCT image dataset for further processing within the system;
d) processing the three-dimensional OCT image dataset through a GON and MF multi-task detection model to produce an AI-GON-score and an AI-MF-score for the three-dimensional OCT image dataset;
e) comparing the AI-GON-score against a second predetermined value to produce an AI-GON-analysis-result; and
f) comparing the AI-MF-score against a third predetermined value to produce an AI-MF-analysis-result.

13. The system of claim 1, wherein:
the three-dimensional OCT image dataset representing an eye of the patient is a first three-dimensional OCT image dataset representing a right eye of the patient;
the image quality assessment rating is a first image quality assessment rating;
the AI-GON-score is a first AI-GON-score;
the AI-MF-score is a first AI-MF-score;
the AI-GON-analysis-result is a first AI-GON-analysis-result;
the AI-MF-analysis-result is a first AI-MF-analysis-result; and
wherein the instructions when executed further repeat steps a)-f) with respect to a second three-dimensional OCT image dataset representing a left eye of the patient, thus producing a second image quality assessment rating, a second AI-GON-score, a second AI-MF-score, a second AI-GON-analysis-result, and a second AI-MF-analysis-result.

14. The system of claim 12, the instructions when executed further performing the following additional steps:
g) reporting the image quality assessment rating, the AI-GON-score, the AI-MF-score, the AI-GON-analysis-result, and the AI-MF-analysis-result.

15. The system of claim 13, the instructions when executed further performing the following additional steps:
g) reporting the first image quality assessment rating, the first AI-GON-score, the first AI-MF-score, the first AI-GON-analysis-result, and the first AI-MF-analysis-result; and
h) reporting the second image quality assessment rating, the second AI-GON-score, the second AI-MF-score, the second AI-GON-analysis-result, and the second AI-MF-analysis-result.

16. The system of claim 15, the instructions when executed further performing the following additional steps:
i) producing a referral-triage suggestion based on any combination of the first image quality assessment rating, the first AI-GON-score, the first AI-MF-score, the first AI-GON-analysis-result, the first AI-MF-analysis-result, the second image quality assessment rating, the second AI-GON-score, the second AI-MF-score, the second AI-GON-analysis-result, and the second AI-MF-analysis-result, respectively.

17. The system of claim 16, the instructions when executed further performing the following additional steps:
j) producing a clinical management suggestion based on the referral-triage suggestion.

18. The system of claim 17, the instructions when executed further performing the following additional steps:
k) comparing one of the image quality assessment rating, the first image quality assessment rating, or the second image quality assessment rating against the first predetermined value and failing to confirm sufficient image quality of the respective three-dimensional OCT image dataset for further processing within the system, thus producing an ungradable image dataset;
l) obtaining, from the OCT scanner, a replacement three-dimensional OCT image dataset;
m) replacing the ungradable image dataset with the replacement three-dimensional OCT image dataset; and
n) repeating steps b)-f) with respect to the replacement three-dimensional OCT image dataset.

19. A system for rapid three-dimensional artificial intelligence-aided classification of glaucomatous optic neuropathy (GON) and myopic optic disc morphology (MF) from optical coherence tomography (OCT) scans, the system comprising:
a user interface subsystem;
an information management subsystem; and
an artificial intelligence (AI) image analysis subsystem comprising:
a pre-diagnosis image quality assessment model, and
a GON and MF multi-task detection model;
wherein the pre-diagnosis image quality assessment model comprises a three-dimensional squeeze-and-excitation (SE) model based on a ResNeXt architecture and comprising a multiplicity of SE-ResNeXt building blocks;
wherein the three-dimensional SE model comprises batch normalization, global average pooling, convolution, and averaging;
wherein the three-dimensional SE model is configured and adapted to produce an output comprising a confirmation of image quality sufficient for reliable operation of the GON and MF multi-task detection model; and
wherein the GON and MF multi-task detection model comprises a shared feature extraction module configured and adapted to deliver a respective input to each of a GON classification module and an MF detection module.

20. The system of claim 19, wherein the shared feature extraction module comprises convolution, batch normalization, a multiplicity of residual units, and global average pooling;
wherein the GON classification module, and the MF detection module each, respectively, comprise a fully connected softmax layer;
wherein the GON and MF multi-task detection model utilizes ResNet-37 in a 3D version;
wherein the pre-diagnosis image quality assessment model comprises an irrelevancy reduction mechanism and a self-attention mechanism; and the GON and MF multi-task detection model is trained using a multi-task learning paradigm.
